(19) United States
(12) Patent Application Publication
(10) Pub. No.: US 2008/0024720 A1
(43) Pub. Date: Jan. 31, 2008

(54) Road Safety

(76) Inventor: Matthew Emmerson Allen, Arundel (GB)

Correspondence Address: Matthew Emmerson Allen, The Lodge, 2 Ford Road, Arundel, West Sussex, ENG, BN18 9EA, GB

(21) Appl. No.: 11/791,408

(22) PCT Filed: Nov. 30, 2005
(86) PCT No.: PCT/GB05/04580

§ 371 Date: May 21, 2007

(30) Foreign Application Priority Data
Nov. 30, 2004 (GB) 0426171.5

(51) Int. Cl. A61B 3/00

(52) U.S. Cl. 351/203

(57) ABSTRACT

The invention relates to a vehicle occupant sight testing assembly. The vehicle occupant sight testing assembly comprises a vehicle stopping area on or adjacent a road, a sight testing means (12) on or adjacent the road, and a vehicle barrier (14) arranged adjacent the vehicle stopping area.

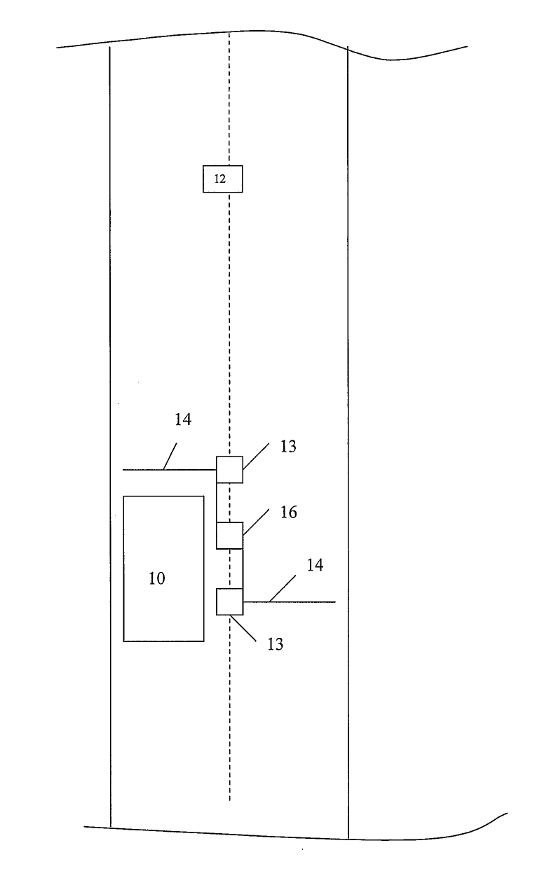

Poor driver eyesight causes a lot of road accidents.

According to the invention there is provided a vehicle occupant sight testing assembly comprising, a vehicle stopping area on or adjacent a road, and a sight testing means on or adjacent the road.

In this way, when a vehicle stops in the vehicle stopping area the sight testing means can be used to test the occupant's (e.g. the driver's) eyesight.

Preferably the sight testing means able to communicate results and/or notice to occupant. Preferably the sight testing means able to communicate results to vehicle authority such as DVLA or police. Most preferably, the sight testing means is in electronic communication with the vehicle authority.

Preferably the vehicle driver sight testing assembly comprises number plate reading technology. Preferably CCTV or the like is in operation to monitor test avoidance/cheating. A warning or penalty can be imposed on drivers who circumvent testing. Driver liability and payment can be dealt with in a similar manner to that used to administer speeding offences. Once number plate is known, and cross check with database of stolen cars is done, the vehicle may be stopped by barrier.

Preferably the vehicle driver sight testing assembly is arranged so that the driver can remain in the car whilst the testing is carried out.

Preferably vehicle occupant sight testing assembly comprises a vehicle barrier arranged adjacent vehicle stopping area. The vehicle barrier is preferably arranged to prevent vehicle passing until test conducted. Moreover, the vehicle barrier may be arranged to prevent vehicle passing if negative sight test results. A warning or penalty can be imposed on drivers who fail the test. As above, driver liability and payment can be dealt with in a similar manner to that used to administer speeding offences.

Preferably the vehicle driver sight testing assembly is arranged as a toll or the like. In this way, the driver only needs to stop once.

Testing assembly may comprise breath analysis equipment. Negative testing can cause barrier to prevent vehicle passing.

Vehicle 10

Sight test means 12

Barrier control means 13

Barrier 14

Control unit 16

Sight test 12 may be set at a certain distance from the vehicle/person 10, which may vary according to the relevant state/country, so as to closely simulate the eyesight test applicable to the relevant state/country.

In another embodiment of the invention, the testing assembly has means for testing another driver parameter, such as reflexes. For example, a reflex test assembly has a display screen, ordinarily blank, and onto which are periodically introduced images of objects. The occupant can then press a button or the like to show that they have seen the object, and the time taken to respond to the appearance of the object can be measured. If the occupant fails the reflex test, the occupant may be allowed to pass the first barrier. However, vehicle is not allowed to pass a second barrier (not shown for simplicity) further down the road by say 100 m. The vehicle is forced to stop in a lay-by or the like between first and second barrier. In this way, the failing driver does not stop traffic flowing. The vehicle of the occupant may be allowed to pass the second barrier only after a set time, e.g. 15 minutes, thereby allowing the driver time to take a break. Food and drink facilities can be dispensed from the assembly to nourish the driver. The assembly may instruct lifting of the second barrier only after the number plate of the vehicle is read and a certain time has elapsed. A ticket or the like may be dispensed to inform the user when they are free to move past the second barrier.

Suitable sensors sense the presence of the vehicle near the barrier. Sensor may cause test to initiate. Sensor can be a load sensor, positioned in a position on which wheels of vehicle can lie in use. The testing assembly can then operate relatively autonomously with occupant intervention to respond to prompts. The testing assembly may comprise an array of buttons or touch pads corresponding to the letters of the alphabet. In use, if the driver sees a letter they can touch the pad corresponding to the letter. The assembly will give a limited amount of time, say ten seconds per part of the test, to input what they see.

Although CCTV records avoidance of test, blocking means can be arranged adjacent barrier 14 so as to form a continuous blockage sufficient to prevent a vehicle passing around the barrier 14 and down the road.

The sight testing assembly may comprise means for calculating driver eye prescription. The assembly may comprise a stock of corrective eyewear such as lenses and/or glasses. These can be dispensed to occupants where required. The occupant can also input their prescription to acquire lenses.

The assembly can have a means to receive payment for lenses or other items or a penalty payment.

A second sight testing assembly can be arranged up the road and a third can be arranged down the road. Knowing the distance and time to drive between assemblies, the assembly can calculate speed between the second and first. If that exceeds the speed limit action can be taken, e.g. dispensing warning and/or fine. Assembly may send signal to engine management unit of vehicle to actively limit speed of vehicle. If sight is failing, drier may be instructed to or forced to limit speed between first and third assembly.

Appropriate shielding and/or lighting can be arranged near sight test means so as to ensure visibility in all weather and times.

According to a second aspect of the invention there is provided a vehicle occupant sight testing assembly comprising vehicle stopping means, such as a movable barrier, and a sight testing means, the assembly being arranged to trigger operation of vehicle stopping means following test.

Vehicle stopping means may alternatively be a traffic light or the like.

In use adjacent a road, the results of the test may be used to initiate a signal to cause vehicle stopping means to change configuration, e.g. to a configuration in which vehicle is permitted to pass or remains stopped.

The vehicle occupant sight testing assembly may have the features set out in relation to the first aspect of the invention.

Assembly may have vehicle sensor, which may cause test to initiate. Vehicle sensor may be a load sensor, positioned in a position on which wheels of vehicle can lie in use.

According to a third aspect of the invention there is provided a vehicle occupant sight testing assembly comprising a vehicle sensor and/or a sight testing means.

The vehicle occupant sight testing assembly may have the features set out in relation to the first or second aspect of the invention.

Although not drawn, the invention relates to any complimentary combination of features possible from those set out above, including implied method steps.

1. A vehicle occupant sight testing assembly comprising a vehicle occupant sight testing means arranged on or adjacent the road and a vehicle stopping area on or adjacent a road.

2. A vehicle occupant sight testing assembly according to claim 1, wherein the sight testing means is able to communicate results and/or notice to a vehicle occupant.

3. A vehicle occupant sight testing assembly according to claim 1, wherein the sight testing means is able to communicate results to vehicle authority, such as driver and vehicle licensing agency or police.

4. A vehicle occupant sight testing assembly according to claim 3, wherein the sight testing means is in electronic communication with the vehicle authority.

5. A vehicle occupant sight testing assembly according to claim 1, wherein the vehicle driver sight testing assembly comprises number plate reading technology.

6. A vehicle occupant sight testing assembly according to claim 1, wherein the assembly comprises CCTV or the like which can be operated to monitor test avoidance/cheating.

7. A vehicle occupant sight testing assembly according to claim 1, wherein a warning or penalty can be imposed on drivers who circumvent testing.

8. A vehicle occupant sight testing assembly according to claim 1, wherein the vehicle driver sight testing assembly is arranged so that the driver can remain in the car whilst the testing is carried out.

9. A vehicle occupant sight testing assembly according to claim 1, wherein vehicle occupant sight testing assembly comprises a vehicle barrier arranged adjacent vehicle stopping area.

10. A vehicle occupant sight testing assembly according to claim 9, wherein the vehicle barrier is arranged to prevent vehicle passing until test conducted.

11. A vehicle occupant sight testing assembly according to claim 9, wherein the vehicle barrier is arranged to prevent vehicle passing if negative sight test results.

12. A vehicle occupant sight testing assembly according to claim 1, wherein warning or penalty can be imposed on drivers who fail the test.

13. A vehicle occupant sight testing assembly according to claim 1, wherein the vehicle driver sight testing assembly is arranged at a toll or the like.

14. A vehicle occupant sight testing assembly comprising a sight testing means to be arranged on or adjacent a road, a vehicle stopping area on or adjacent a road, and a vehicle barrier arranged adjacent the vehicle stopping area.

15. A method of testing the sight of a vehicle occupant, the method comprising using a vehicle occupant sight testing assembly arranged on or adjacent a road.
